(19) United States
(12) Patent Application Publication
(10) Pub. No.: US 2024/0331342 A1
(43) Pub. Date: Oct. 3, 2024

(54) SYSTEM AND METHOD FOR DETERMINING AN ORTHODONTIC OCCLUSION CLASS

(71) Applicant: ORTHODONTIA VISION INC., Mont-Royal (CA)

(72) Inventors: Charles Fallaha, Laval (CA); Normand Bach, Mont-Royal (CA)

(21) Appl. No.: 18/576,192

(22) PCT Filed: Jul. 5, 2022
(86) PCT No.: PCT/CA2022/051058

§ 371 Date: Jan. 3, 2024

Related U.S. Application Data
(60) Provisional application No. 63/203,030, filed on Jul. 6, 2021.

(51) Int. Cl. G06V 10/26; G06V 10/77; G06V 10/82

(52) U.S. Cl. CPC G06V 10/26; G06V 10/7715; G06V 10/82

(57) ABSTRACT

Systems and methods are provided for determining an occlusion class indicator corresponding to an occlusion image. This can include acquiring the occlusion image of an occlusion of a human subject by an image capture device, applying one or more computer-implemented occlusion classification neural networks to the occlusion image to determine the class indicator of the occlusion of the human subject. The occlusion classification neural networks are trained for classification using an occlusion training dataset including a plurality of occlusion training examples being pre-classified into one three occlusion classes, each class being attributed a numerical value. The occlusion class indicator determined by the occlusion classification neural network includes a numeric value within a continuous range of values that can be bounded by the values corresponding to the second and third occlusion classes.

### TECHNICAL FIELD

The present disclosure generally relates to a method for determining an orthodontic occlusion class based on applying computer-implemented classification neural network(s) to orthodontic image(s) of a human subject and, more particularly, applying the neural network(s) to determine an occlusion class indicator in the form of a numerical value within a continuous range of values, the occlusion class indicator providing an indication of a class of the orthodontic occlusion.

### BACKGROUND

In dental medicine, images of a patient's occlusion, in conjuncture with the clinical exam, radiographic images and dental models, assist in the diagnosis and help to determine a treatment plan for the patient. Images of the patient's dental occlusion are typically taken in a clinical setting by an assistant or a hygienist. The images of the dental occlusion are then reviewed by the dentist or the orthodontist, who will then confirm the diagnosis.

Part of the diagnosis include an identification of the posterior occlusion class of the patient's (right and left) as well as an identification of the patient's anterior occlusion. The treatment plan can include one or more options chosen from no treatment required, use of a corrective device such as braces, growth modification appliances or surgery, minor surgery, or a major surgery.

The need for a subject to be in a clinical setting and for the professional's involvement significantly reduces a normal person's ability to access diagnosis and treatment for their orthodontic malocclusion.

### SUMMARY

According to an aspect, there is provided a method for determining at least one occlusion class indicator corresponding to at least one occlusion image, the method comprising: acquiring the at least one occlusion image of an occlusion of a human subject by an image capture device; applying at least one computer-implemented occlusion classification neural network to the at least one occlusion image to determine the at least one occlusion class indicator of the occlusion of the human subject, the at least one occlusion classification neural network being trained for classification using at least one occlusion training dataset, each given at least one occlusion training dataset including a plurality of occlusion training examples being pre-classified into one of at least: a first occlusion class, being attributed a first numerical value for the given occlusion type training dataset; a second occlusion class, being attributed a second numerical value for the given occlusion type training dataset; a third occlusion class, being attributed a third numerical value for the given occlusion type training dataset, wherein the first numerical value is between the second numerical value for the given occlusion type training dataset and the third numerical value for the given occlusion type training dataset; each occlusion training example comprising: a respective training occlusion image, being input data; and its respective numerical value, being output data; wherein the at least one occlusion class indicator of the occlusion of the human subject determined by the at least one computer-implemented occlusion classification neural network includes at least one numerical output value within a continuous range of values having the second numerical value as a first bound and the third numerical value as a second bound.

In some embodiments, the image capture device is comprised in a mobile device running a mobile application.

In some embodiments, the at least one occlusion classification neural network comprises an anterior occlusion classification neural network; wherein the at least one occlusion training dataset comprises an anterior occlusion training dataset for training the anterior occlusion classification neural network, the plurality of occlusion training examples of the anterior occlusion training dataset being pre-classified into at least: an ordinary anterior occlusion class, representing the first occlusion class and being attributed the first numerical value for the anterior occlusion training dataset; an open bite occlusion class, representing the second occlusion class and being attributed the second numerical value for the anterior occlusion training dataset; a deep bite occlusion class, representing the third occlusion class and being attributed the third numerical value for the anterior occlusion training dataset; and wherein the at least one occlusion class indicator of the occlusion of the human subject includes an anterior occlusion numerical output value determined by the anterior occlusion classification neural network, the anterior occlusion numerical output value being in the continuous range of values having the second numerical value for the anterior occlusion training dataset as a first bound and the third numerical value for the anterior occlusion training dataset as a second bound.

In some embodiments, the at least occlusion classification neural network comprises a posterior occlusion classification neural network; wherein the at least one occlusion training dataset comprises a posterior occlusion training dataset for training the posterior occlusion classification neural network, the plurality of occlusion training examples of the posterior occlusion training dataset being pre-classified into at least: a class I posterior occlusion class, representing the first occlusion class and being attributed the first numerical value for the posterior occlusion training dataset; a class II posterior occlusion class, representing the second occlusion class and being attributed the second numerical value for the posterior occlusion training dataset; a class III posterior occlusion class, representing the third occlusion class and being attributed the third numerical value for the posterior occlusion training dataset; wherein the at least one occlusion class indicator of the occlusion of the human subject includes a posterior occlusion numerical output value determined by the posterior occlusion classification neural network, the posterior occlusion numerical output value being in the continuous range of values having the second numerical value for the posterior occlusion training dataset as a first bound and the third numerical value for the posterior occlusion training dataset as a second bound.

In some embodiments, the at least one occlusion classification neural network comprises an anterior occlusion classification neural network and a posterior occlusion classification neural network; wherein the at least one occlusion training dataset comprises an anterior occlusion training dataset for training the anterior occlusion classification neural network and a posterior occlusion training dataset for training the posterior occlusion classification neural network; wherein the plurality of occlusion training examples of the anterior occlusion training dataset is pre-classified into at least: an ordinary anterior occlusion class, representing the first occlusion class and being attributed the first numerical value for the anterior occlusion training dataset; an open bite occlusion class, representing the second occlusion class and being attributed the second numerical value for the anterior occlusion training dataset; a deep bite occlusion class, representing the third occlusion class and being attributed the third numerical value for the anterior occlusion training dataset; and wherein the at least one occlusion class indicator of the occlusion of the human subject includes an anterior occlusion numerical output value determined by the anterior occlusion classification neural network, the anterior occlusion numerical output value being in a first continuous range of values having the second numerical value for the anterior occlusion training dataset as a first bound and the third numerical value for the anterior occlusion training dataset as a second bound; wherein the plurality of occlusion training examples of the posterior occlusion training dataset is pre-classified into at least: a class I posterior occlusion class, representing the first occlusion class and being attributed the first numerical value for the posterior occlusion training dataset; a class II posterior occlusion class, representing the second occlusion class and being attributed the second numerical value for the posterior occlusion training dataset; a class III posterior occlusion class, representing the third occlusion class and being attributed the third numerical value for the posterior occlusion training dataset; wherein the at least one occlusion class indicator of the occlusion of the human subject includes a posterior occlusion numerical output value determined by the posterior occlusion classification neural network, the posterior occlusion numerical output value being in the continuous range of values having the second numerical value for the posterior occlusion training dataset as a first bound and the third numerical value for the posterior occlusion training dataset as a second bound.

In some embodiments, the at least one occlusion image of the human subject comprises a left posterior occlusion image, a right posterior occlusion image, and an anterior occlusion image; wherein the posterior occlusion classification neural network is applied to the left posterior occlusion image to determine a left posterior occlusion numerical output value; wherein the posterior occlusion classification neural network is applied to the right posterior occlusion image to determine a right posterior occlusion numerical output value; and wherein the anterior occlusion classification neural network is applied to the anterior occlusion image to determine the anterior occlusion numerical output value.

In some embodiments, the at least one occlusion class indicator further comprises an interpolation of at least two output values selected from the group consisting of the left posterior occlusion numerical output value, the right posterior occlusion numerical output value and the anterior numerical output value.

In some embodiments, the method further comprises cropping and normalizing the at least one occlusion image of the occlusion of the human subject prior to applying the at least one computer-implemented occlusion classification neural network thereto.

In some embodiments, cropping the at least one occlusion image is performed semi-automatically using at least one overlaid mask.

In some embodiments, acquiring the at least one occlusion image comprises: displaying a live view of a first scene and a left posterior occlusion mask overlaid on the live view of the first scene; in response to a first capture command, capturing a first image corresponding to the first scene, the first image being the left posterior occlusion image of the at least one occlusion image of the occlusion of the human subject; displaying a live view of a second scene and a right posterior occlusion mask overlaid on the live view of the second scene; in response to a second capture command, capturing a second image corresponding to the second scene, the second image being the right posterior occlusion image of the at least one occlusion image of the occlusion of the human subject; displaying a live view of a third scene and an anterior occlusion mask overlaid on the live view of the third scene; and in response to a third capture command, capturing a third image corresponding to the third scene, the third image being the anterior occlusion image of the at least one occlusion image of the occlusion of the human subject.

In some embodiments, the at least one computer-implemented occlusion classification neural network comprises at least one radial basis function neural network.

In some embodiments, applying the at least one radial basis function neural network comprises extracting a feature vector from each of the at least one occlusion image.

In some embodiments, extracting the feature vector comprises applying a principal component analysis to each of the at least one occlusion image.

In some embodiments, the at least one radial basis function neural network is configured to receive the feature vector.

In some embodiments, the feature vector has between approximately 25 features and approximately 100 features.

In some embodiments, the at least one radial basis function neural network has between approximately 10 centres and approximately 20 centres.

In some embodiments, the method further comprises determining that a given one of the at least one occlusion image is an inappropriate occlusion image based on the given occlusion image being greater than a threshold distance from each of the centres.

According to another aspect, there is provided a use of the method as described above in diagnosing an orthodontic malocclusion.

According to a further aspect, there is provided a use of the method as described above in determining a treatment for an orthodontic malocclusion.

According to yet another aspect, there is provided a system for determining at least one occlusion class indicator, the system comprising: at least one data storage device storing executable instructions; at least one processor coupled to the at least one storage device, the at least one processor being configured for executing the instructions and for performing the method as described above.

According to yet a further aspect, there is provided a computer program product comprising a computer readable memory storing computer executable instructions thereon that when executed by a computer perform the method as described above.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 illustrates a schematic diagram of the high-level modules of a computer-implemented occlusion classification system for classifying an orthodontic occlusion according to an example embodiment;

FIG. 2 illustrates a schematic diagram of the architecture of one occlusion classification neural network according to one example embodiment;

FIG. 3 illustrates a representation of a Gaussian-type function of an exemplary neuron of a classification neural network having RBF architecture;

FIG. 4 illustrates a clustering of the centres of three neurons of the classification neural network;

FIG. 5A illustrates a detailed schematic diagram of the computer-implemented occlusion classification system according to one example embodiment;

FIG. 5B showing an exemplary decision table for interpolating between occlusion classes for determining a recommended treatment;

FIG. 6 illustrates a flowchart showing the operational steps of a method for classifying an orthodontic occlusion according to one example embodiment:

FIG. 7 illustrates a flowchart showing the detailed operational steps of a method for classifying an orthodontic occlusion according to one example embodiment;

FIG. 8 illustrates a user interface for capturing occlusion images for a human subject according to one example embodiment;

FIGS. 9a, 9b and 9c show screenshots of the user interface while in three camera modes for capturing a right posterior occlusion image, a left posterior occlusion image, and an anterior occlusion image according to one example embodiment;

FIG. 10 shows the flowchart of the operational steps of a method for capturing occlusion images according to one example embodiment;

FIG. 11 is a chart showing the posterior occlusion machine learning error reduction for an experimental implementation of a posterior occlusion classification neural network;

FIG. 12 is a chart showing the anterior occlusion machine learning error reduction for an experimental implementation of an anterior occlusion classification neural network;

FIG. 13 shows a first posterior occlusion image classified by the experimentally implemented posterior occlusion classification neural network;

FIG. 14 shows a second posterior occlusion image classified by the experimentally implemented posterior occlusion classification neural network.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art, that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

One or more systems described herein may be implemented in computer programs executing on processing devices, each comprising at least one processor, a data storage system (including volatile and non-volatile memory and/or storage elements), at least one input device, and at least one output device. The term “processing device” encompasses computers, servers and/or specialized electronic devices which receive, process and/or transmit data. “Processing devices” are generally part of “systems” and include processing means, such as microcontrollers and/or microprocessors, CPUs or are implemented on FPGAs, as examples only. For example, and without limitation, the processing device may be a programmable logic unit, a mainframe computer, server, and personal computer, cloud based program or system, laptop, personal data assistance, cellular telephone, smartphone, wearable device, tablet device, video game console, or portable video game devices.

Each program is preferably implemented in a high-level procedural or object-oriented programming and/or scripting language to communicate with a computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein. In some embodiments, the system may be embedded within an operating system running on the programmable computer.

Furthermore, the system, processes and methods of the described embodiments are capable of being distributed in a computer program product comprising a computer readable medium that bears computer-usable instructions for one or more processors. The computer-usable instructions may also be in various forms including compiled and non-compiled code.

The processor(s) are used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory is of course chosen according to the desired use, whether it should retain instructions, or temporarily store, retain or update data. Steps of the proposed method are implemented as software instructions and algorithms, stored in computer memory and executed by processors.

“Occlusion classification neural network” referred to herein comprise one or several computer-implemented machine learning algorithms that can be trained, using training data. New data can thereafter be inputted to the neural network which predicts or estimates an output according to parameters of the neural network, which were automatically learned based on patterns found in the training data.

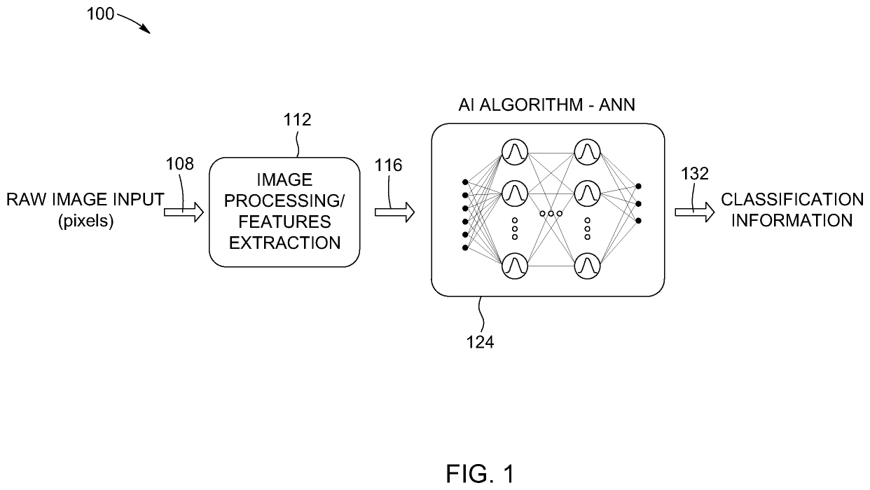

FIG. 1 illustrates a schematic diagram of the high-level modules of a computer-implemented occlusion classification system 100 for classifying an orthodontic occlusion according to one example embodiment.

The occlusion classification system 100 receives at least one occlusion image 108 of an occlusion of a human subject, for instance an orthodontic patient or potential patient. As described elsewhere herein, for a given subject, a set of occlusion images 108 may be received, this set including a left posterior occlusion image, right posterior occlusion image and an anterior occlusion image. The occlusion classification system 100 may further include an image processing/feature extraction module 112 configured to carry out image processing steps to the occlusion image(s) 108 and extract features 116 from the occlusion image(s). A first set of features 116 can be generated for the left posterior occlusion image, a second set of features 116 can be generated for the right posterior occlusion image, and a third set of features 116 can be generated for the anterior occlusion image.

The occlusion classification system 100 further includes at least one computer-implemented occlusion classification neural network 124 that receives the extracted features 116. When applied to the received extracted features, the at least one computer-implemented occlusion classification neural network 124 determines at least one occlusion class indicator 132 for the occlusion of the subject. The class indicator 132 provides an indication of a class of the orthodontic occlusion of the subject and the indication can be further used to automatically determine a treatment plan for the subject. As described elsewhere herein, an appropriate corresponding computer-implemented occlusion classification neural network 124 is applied to each occlusion image 108 (ex: in the form of its corresponding set of extract features 116) and a corresponding occlusion class indicator 132 for that occlusion image 108 is determined by the neural network 124. For example, for the left posterior occlusion image 108, its corresponding computer-implemented occlusion classification neural network 124 is applied to it (ex: in the form of the extracted features set 116 for that image) and a left posterior occlusion class indicator 132 is determined. For the right posterior occlusion image 108, its corresponding computer-implemented occlusion classification neural network 124 is applied to it (ex: in the form of the extracted features set 116 for that image) and a right posterior occlusion class indicator 132 is determined. Similarly, for the anterior occlusion image 108, its corresponding computer-implemented occlusion classification neural network 124 is applied to it (ex: in the form of the extracted features set 116 for that image) and an anterior occlusion class indicator 132 is determined. According to one example embodiment, and as described elsewhere herein, a same posterior occlusion classification neural network 124 is applied to both the left posterior occlusion image and the right posterior occlusion image to determine the left posterior occlusion class and the right posterior occlusion class and an anterior occlusion classification neural network 124 is applied to the anterior occlusion image 108.

The at least one occlusion classification neural network 124 is trained by machine learning for classification using at least one occlusion training dataset. More particularly, each occlusion classification neural network 124 is trained using a corresponding occlusion training dataset. Each occlusion training dataset includes a plurality of occlusion training examples that have been pre-classified. Each training example includes at least a training occlusion image and an occlusion class of that training occlusion image as defined during pre-classification. When used for training by machine learning, the training occlusion images of the training examples are used as the input data and the occlusion classes of the training examples are used as the output data.

At least three occlusion classes are defined. Each class of the training examples is attributed a respective numerical value. The numerical value for each given class relative to the numerical value of other classes is representative of where that given class falls within a spectrum of occlusion conditions relative to where the other classes fall within the spectrum of occlusion conditions. More particularly, a first occlusion class represents an ordinary, or normal, condition that falls at an intermediate position within the spectrum of occlusion conditions and is attributed a first numerical value that is representative of the intermediate position. A second occlusion class represents a first occlusion condition that deviates in a first direction along the spectrum from the ordinary condition and is attributed a second numerical value that is representative of this first position of deviation. The second occlusion class can represent a position along the spectrum that is towards a first end of the spectrum of occlusion conditions. The third occlusion class can represent a second occlusion condition that deviates in a second direction along the spectrum from the ordinary condition, this second direction being opposite the first direction of deviation. The third occlusion class is attributed a third numerical value that is representative of this second position of deviation. The third occlusion class can represent a position along the spectrum that is towards a second end of the spectrum of occlusion conditions, the second end being opposite to the first end.

The relative values of the first, second and third numerical values are representative of the relative positions of each respective occlusion class along the spectrum of occlusion conditions. More particularly, the first numerical value attributed to the first occlusion class lies between the second numerical value and the third numerical value, thereby representing that the second and third occlusion classes are at opposite ends of the spectrum and the first occlusion class is an intermediate condition.

According to one example embodiment, the first occlusion class is attributed the first numerical value “1.0”, the second occlusion class is attributed the second numerical value “2.0” and the third occlusion class is attributed the third numerical value “0.0”. It will be appreciated that the first numerical value “1.0” lies between the second numerical value “2.0” and the third numerical value “0.0”. The decimal representation (i.e. “X.0”) indicates numerical values other than first, second, and third numerical values can possibly be used to represent other occlusion conditions that fall within the spectrum, such as between the second numerical value and the third numerical value but other than the first numerical value (ex: values such as “0.3” or “1.7”). This more specific value can be indicative how the given condition relates to the first occlusion class, the second occlusion class and the third occlusion class.

The at least one computer-implemented occlusion classification neural network 124 is trained by machine learning using the occlusion training dataset having the above-described training examples so that it can predict, for a given to-be-classified occlusion image, an occlusion class indicator that indicates the occlusion class of that occlusion image. The predicted occlusion class indicator also takes the form of a numerical output value. This numerical output value is within a continuous range of values having the second numerical value as a first bound, which may be an upper bound, and the third numerical value as a second bound, which may be a lower bound. Since this range is continuous, this numerical output value as the occlusion class indicator for the given occlusion image can have a value other than the first numerical value, the second numerical value or the third numerical value. Moreover, the numerical output value relative to the first, second and third numerical values is intended to be predictive of where the occlusion image falls within the spectrum of possible occlusion conditions.

It was observed that although orthodontic occlusion conditions are classified into discrete occlusion classes, the possible conditions actually lie on a spectrum of conditions. This variance is typically accounted for by the orthodontic professional when making their assessment of the treatment plan for a given subject.

By attributing numerical values to occlusion classes of the training examples of the training dataset and further training the occlusion classification neural network by machine learning to predict the occlusion class indicator as the numerical output value within the continuous range of values, the prediction that is made captures this reality that the possible occlusion conditions lie on the spectrum of conditions.

According to one example embodiment, and as described elsewhere herein, the at least one occlusion classification neural network 124 includes an anterior occlusion classification neural network and a posterior occlusion classification neural network. The at least one occlusion training dataset includes an anterior occlusion training dataset that is used for training the anterior occlusion classification neural network by machine learning. The occlusion training examples of the anterior occlusion training dataset are pre-classified into the at least three occlusion classes, which are:
an ordinary, or normal, anterior occlusion class—representing the first occlusion class and being attributed the first numerical value for the anterior occlusion training dataset (ex: value “1.0”);
an open bite occlusion class—representing the second occlusion class and being attributed the second numerical value for the anterior occlusion training dataset (ex: value “2.0”);
a deep bite occlusion class, representing the third occlusion class and being attributed the third numerical value for the anterior occlusion training dataset (ex: value “0.0”).

After training by machine learning, the trained anterior occlusion classification neural network is operable to receive an image of an anterior occlusion of a subject and to determine an anterior occlusion numerical output value. This numerical output value can be any value in the continuous range of values having the second numerical value for the anterior occlusion training dataset as its first (upper) bound and the third numerical value for the anterior occlusion training dataset as a second (lower) bound.

The at least one occlusion type training dataset includes a posterior occlusion classification neural network that is used for training the posterior occlusion classification neural network by machine learning. The occlusion training examples of the posterior occlusion training dataset are pre-classified into the at least three occlusion classes, which are:
a class I posterior occlusion class—representing the first occlusion class and being attributed the first numerical value for the posterior occlusion training dataset (ex: value “1.0”);
a class II posterior occlusion class—representing the second occlusion class and being attributed the second numerical value for the posterior occlusion training dataset (ex: value “2.0”);
a class III posterior occlusion class, representing the third occlusion class and being attributed the third numerical value for the posterior occlusion training dataset (ex: value “0.0”).

After training by machine learning, the trained posterior occlusion classification neural network is operable to receive an image of a posterior occlusion of a subject and determine a posterior occlusion numerical output value. This numerical output value can be any value in the continuous range of values having the second numerical value for the posterior occlusion training dataset as its first (upper) bound and the third numerical value for the anterior occlusion training dataset as a second (lower) bound.

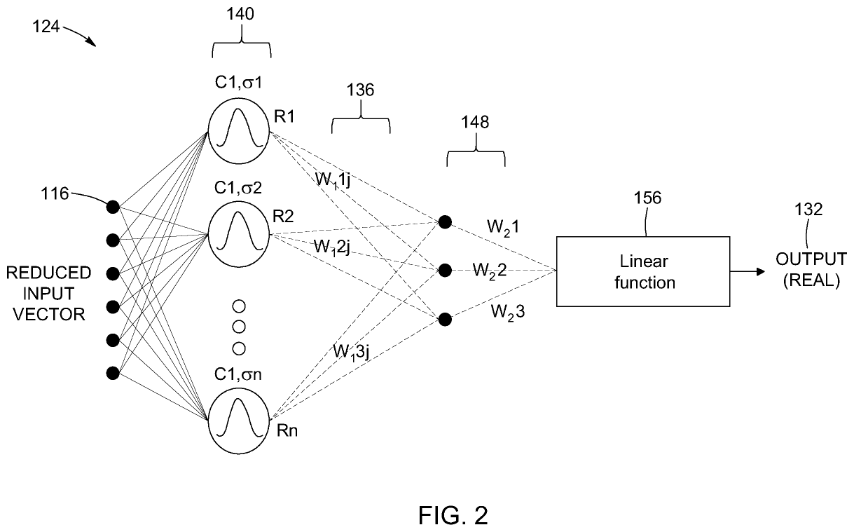

Referring now FIG. 2, therein illustrated is a schematic diagram of the architecture of one occlusion classification neural network 124 according to one example embodiment. According to exemplary embodiments in which more than one occlusion classification neural network 124 is included in the orthodontic classification system 100, each occlusion classification neural network 124 has the architecture illustrated in FIG. 2. Each of the at least one occlusion classification neural network 124 has a radial basis functions (RBF) architecture, which is a compact form of a neural network.

The occlusion classification neural network 124 receives an occlusion image for classification. The occlusion image can be inputted in the form of its extracted feature vector 116. Within the occlusion classification neural network 124 having the RBF architecture, each neuron has the form of a Gaussian-type function with a centre vector and a standard deviation value.

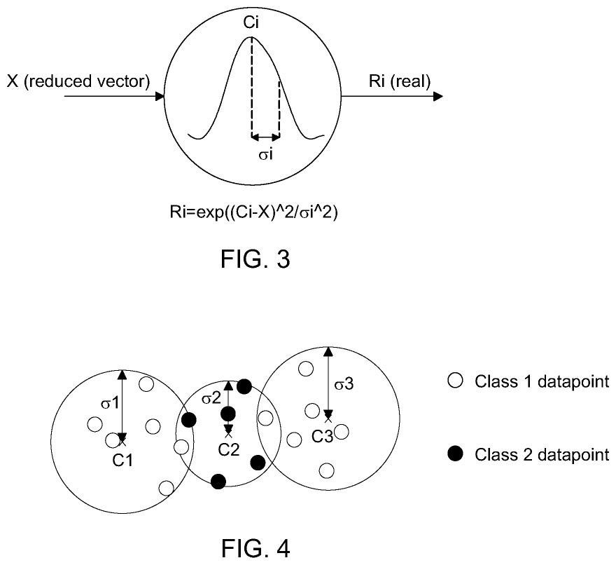

FIG. 3 illustrates a representation of a Gaussian-type function of an exemplary neuron.

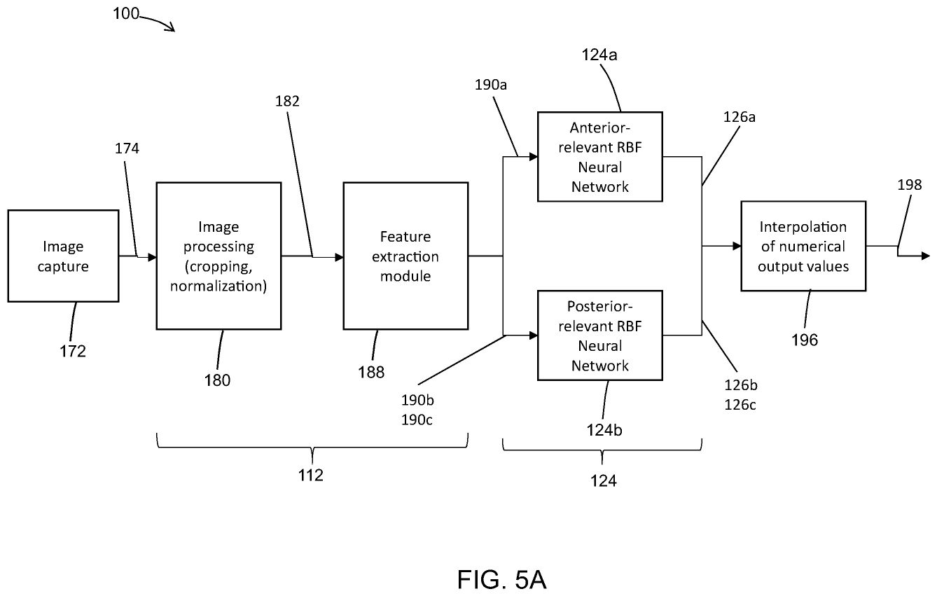

The centre and their respective standard deviation for each of the neurons are initially obtained with a clustering algorithm. This clustering is illustrated in FIG. 4, which shows the centres (C1, C2, C3) and their respective standard deviation (a1, a2, a3). In the illustrated example, Class 1 has 2 centres C1 and C3, and has Class 2 has a single centre C2.

Referring back to FIG. 2, each layer of the occlusion classification neural network 124 having the RBF architecture is linked to an adjacent layer with tuneable weights Wij 136.

According to one example embodiment, the occlusion classification neural network 124 having the RBF architecture is implemented with a single layer 140 of neurons, which are linked to the output layer 148 via the tuneable weights. A linear function 156 is applied to the output layer 148 to produce the output as the numerical output value 132 within the continuous range of values.

In the illustrated example, the output layer has three sublayers corresponding to the three occlusion classes. In other example implementations in which more classes are defined, the output layer 148 may have additional sub-layers. Similarly, additional neurons or layers of neurons can be used.

The initial values of the tuneable weights are selected so as to reduce offset (or bias) in architecture of the neural network.

The occlusion classification neural network 124 having the RBF architecture is trained by machine learning using an appropriate training dataset (ex: the anterior occlusion training dataset or the posterior occlusion training dataset, as appropriate). Various machine learning methods can be used for training. According to one example embodiment, a gradient descent algorithm is used for the machine learning. The gradient descent algorithm can act simultaneously to adjust the centres of the neurons, the standard deviations of the neurons and the weights Wij.

According to one exemplary embodiment, the occlusion classification neural network 124 having the RBF architecture has between approximately 5 to 15 approximately centres in the neuron layer 140. The feature vectors 116 inputted into the neural network can have between approximately 25 features and approximately 100 features.

As described elsewhere herein, it was observed that the occlusion classification neural network 124 having the RBF architecture and a single layer 140 of neurons provided good performance even when trained using a training dataset of a relatively limited image dataset. Due to the lightweight structure of the RBF architecture, the training and implementation also have reasonable hardware and software requirements.

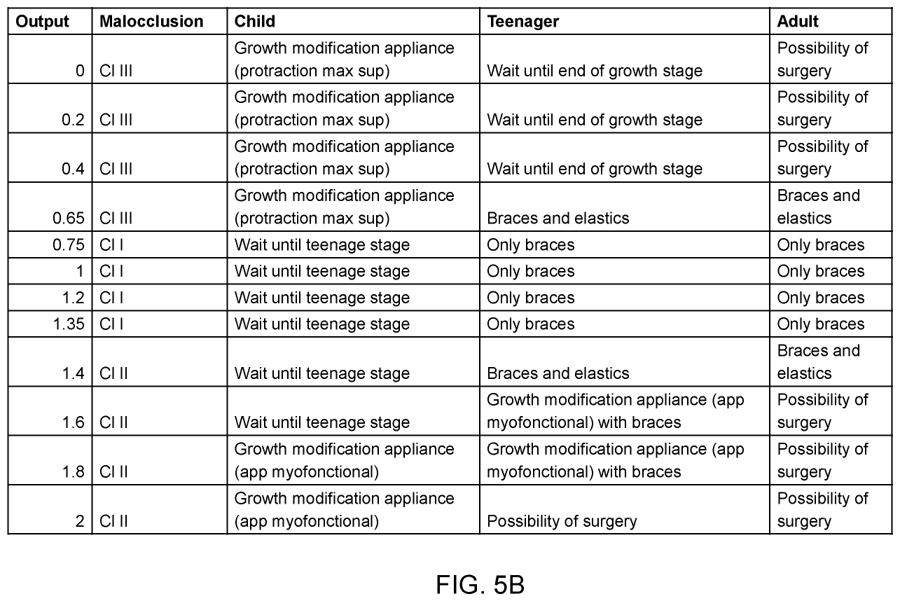

Referring to FIG. 5A, therein illustrated is a detailed schematic diagram of the computer-implemented occlusion classification system 100 according to one example embodiment. An image capture device 172 is used to capture the at least one occlusion image 108 for classification. The image capture device 172 may be the camera of a typical user device (ex: smartphone, tablet, webcam of a computer, etc.) operated by the subject or someone helping the subject. According to one example embodiment, and as illustrated in FIG. 5A, a raw left posterior image, a raw right posterior image and a raw single anterior image 174 are captured as the occlusion images 108 for classification.

The computer-implemented occlusion classification system 100 also includes an image processing module 180, which is part of the image processing/feature extraction module 112. According to one example embodiment, the image processing module 180 may include cropping the captured images (to retain only the image regions corresponding to the subject's occlusion). In some embodiments, cropping the images is a semi-automatic process performed using overlaid masks. An overlaid mask can for instance be a bitmap image of the same size as the image to be cropped wherein each pixel has a value of 1, meaning that the pixel in the image to be cropped is to be kept, or 0, meaning the pixel in the image to be cropped is to be removed. In some embodiments, a person can define an overlaid mask based on a stationary display of the image to be cropped by positioning corners of a polygon overlaid over the image, the pixels inside the area of the polygon being assigned a value of 1 and the pixels outside being assigned a value of 0, then the image processing module 180 can apply the overlaid mask to the image by applying a bitwise and operation on each pixel. In alternative embodiments, a stationary polygon is overlaid over the image, and a person can define an overlaid mask by resizing and translating the image under the polygon. The image processing module 180 may also include normalizing the captured images, which may include normalizing brightness. According to the example embodiment, and as illustrated in FIG. 5, a processed left posterior image, a processed right posterior image and a processed anterior image 182 are outputted by the image processing module 180.

The computer-implemented occlusion classification system 100 also includes the feature extraction module 188, which is also part of the image processing/feature extraction module 112. According to one example embodiment, the feature extraction module 188 is configured to apply principal component analysis to extract the main differentiating features of the image, which provides a reduced feature vector for each inputted image e.g., an anterior vector 190a, a left posterior vector 190b and a right posterior vector 190c. The feature extraction module 188 may also be configured to normalize each feature vector, such as to generate unitary feature vectors. According to the example illustrated in FIG. 5, a left posterior feature vector is determined for the received processed left posterior image, a right posterior feature vector is determined for the received right posterior image and an anterior vector is determined for the received processed anterior image. The feature vector 116 for each image can have between approximately 25 features and approximately 100 features.

As described elsewhere herein, the computer-implemented occlusion classification system 100 includes the at least one computer-implemented occlusion classification neural network 124, which receives the at least one occlusion image 174 in the form of the feature vector 190a-c and outputs the occlusion class indicator 126a-c for each image. According to the example illustrated in FIG. 5, the at least one computer-implemented occlusion classification neural network includes an anterior occlusion classification neural network 124a and a posterior occlusion classification neural network 124b. The anterior occlusion classification neural network 124a receives the anterior vector 190a and outputs the anterior occlusion numerical output value 126a. The posterior occlusion classification neural network 124b is applied to both the left posterior vector 190b and the right posterior vector 190c and respectively outputs a left posterior numerical output value 126b and a right posterior numerical output value 126c.

According to one example embodiment, and as illustrated in FIG. 5A, the classification system 100 further includes an interpolation module 196 that is configured to receive each of the anterior occlusion numerical output value 126a, the left posterior numerical output value 126b and a right posterior numerical output value 126c and to determine, based on these output values, a recommended treatment 198 for the subject. The determination may be based on the individual value one of the continuous-range output values (i.e. a single one of any of the anterior occlusion numerical output value 126a, the left posterior numerical output value 126b and the right posterior numerical output value 126c) and/or the relative or combined values of two or more of the continuous-range output values (i.e. two or more of the anterior occlusion numerical output value 126a, the left posterior occlusion numerical output value 126b and the right posterior occlusion numerical output value 126c). The interpolation module 196 can be implemented as a decision tree. It will be appreciated that the output values each being in a continuous range of possible values allows for a much larger (in theory, unlimited) number of permutations of individual, relative and combined values of the numerical output values, which allows for more dimensions when implementing the decision tree used for determining the recommended treatment 198. When considering relative or combination of output values, a type of inter-class interpolation is implemented. This is in contrast to the limited possibilities if the classification neural networks were configured to classify images into a limited number of discrete occlusion classes (ex: 3 possible classes for each occlusion image), in which cases the number of permutations would be far more limited.

FIG. 5B is a table showing a decision tree implemented by the interpolation module for determining a recommended treatment.

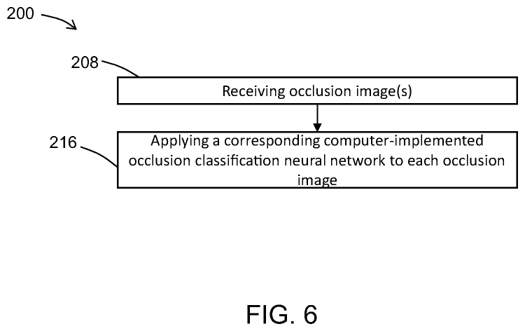

Referring now to FIG. 6, therein illustrated is a flowchart showing the operational steps of a method 200 for classifying an orthodontic occlusion for a given subject according to one example embodiment. At step 208, at least one occlusion image for the subject is received, which can include a left posterior occlusion image, a right posterior occlusion image and an anterior occlusion image.

At step 216, a corresponding computer-implemented occlusion classification neural network is applied to each occlusion image to generate a respective occlusion class indicator in the form of a numerical output value. The neural network can be the at least one occlusion classification neural network 124 described herein according to various example embodiments.

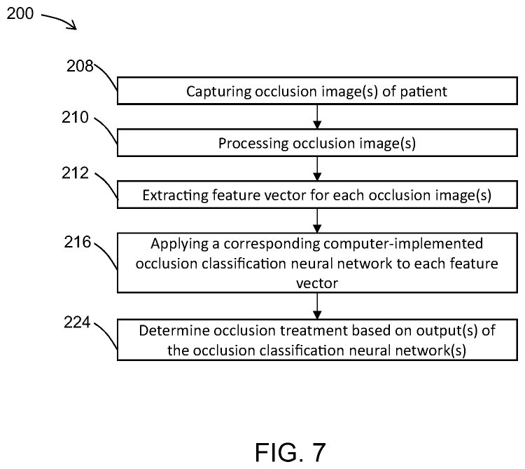

Referring now to FIG. 7, therein illustrated is a flowchart showing detailed operational steps of a method for classifying an orthodontic occlusion according to one example embodiment.

At step 208, the receiving the occlusion image can include capturing the at least one occlusion of the image subject using an image capture device (ex: camera of a smartphone, tablet, webcam or a computer, etc.).

At step 210, each of the captured images are processed. The processing can include the steps as described with reference to image processing module 180.

At step 212, for each of the processed occlusion images, feature extraction is applied to extract a respective feature vector. The feature extraction can be performed as described with reference to feature extraction module 188.

The classification at step 216 is then applied using a corresponding computer-implemented occlusion classification neural network to each feature vector.

At step 224, a recommended occlusion treatment is determined based on an evaluation (ex: interpolation) of the numerical output values outputted from the classification of step 216.

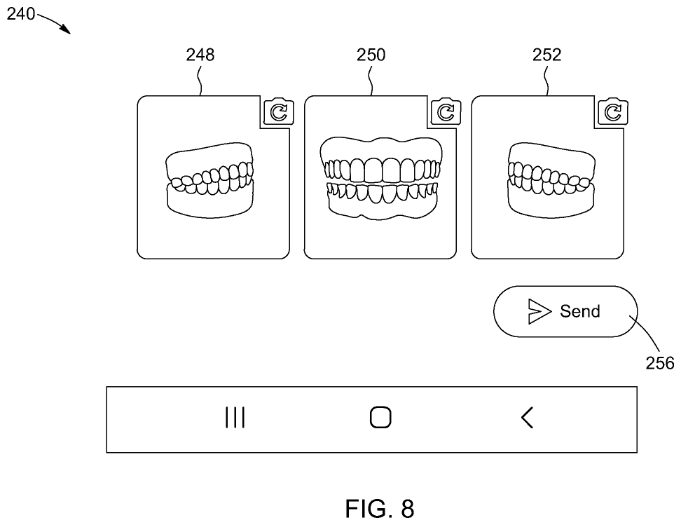

According to one example embodiment, and as described herein, the occlusion image(s) for a given subject can be captured using a camera of a typical user device. The camera can be operated by the subject themselves or by another person helping the subject. A user interactive application, such as mobile application or a desktop software application, can provide a user interface that guides the user in capturing each of a left posterior image, right posterior image and anterior image, while also aiding in ensuring that the captured images are of sufficient quality. FIG. 8 illustrates a user interface 240 that presents a first user selectable icon 248 that leads the user to a first camera mode for capturing a right posterior occlusion image, a second user selectable icon 250 that leads the user to a second camera mode for capturing an anterior occlusion image, and a third user selectable icon 252 that leads the user to a third camera mode for capturing a left posterior occlusion image. A “SEND” option 256 is further made available after the images are captured for transmitting the images for classification.

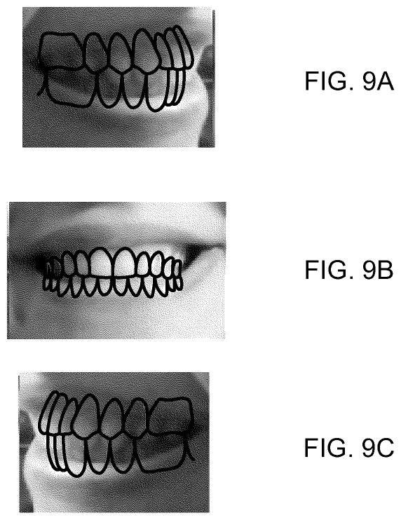

FIG. 9a shows a screenshot while in the first camera mode for capturing a right posterior occlusion image. A live view of a scene captured by the camera is displayed and a right posterior occlusion mask is overlaid on the live view of the first scene. The user can then operate the camera (ex: change orientation, zoom, etc.) so that an image region corresponding to the subject's right posterior occlusion is in alignment with the overlaid right posterior occlusion mask. Upon alignment, the user can then provide a capture command (ex: by depressing a shutter button) to capture an instant image, which is stored as the right posterior occlusion image.

FIG. 9b shows a screenshot while in the second camera mode for capturing an anterior occlusion image. A live view of a scene captured by the camera is displayed and an anterior occlusion mask is overlaid on the live view of the second scene. The user can then operate the camera so that an image region corresponding to the subject's anterior occlusion is in alignment with the overlaid anterior occlusion mask. Upon alignment, the user can provide a second capture command to capture a second instant image, which is stored as the anterior occlusion image.

FIG. 9c shows a screenshot while in the third camera mode for capturing a left posterior occlusion image. A live view of a scene captured by the camera is displayed and a left posterior occlusion mask is overlaid on the live view of the third scene. The user can then operate the camera so that an image region corresponding to the subject's left posterior occlusion is in alignment with the overlaid left posterior occlusion mask. Upon alignment, the user can provide a third capture command to capture a third instant image, which is stored as the left posterior occlusion image.

The use of the overlaid masks aids the user in ensuring proper alignment and orientation to capture the appropriate portions of the subject's occlusion. The use of the overlaid masks also aids in ensuring proper sizing of the occlusion within each occlusion image. The overlaid masks can further define the region of the image to be cropped when processing the image.

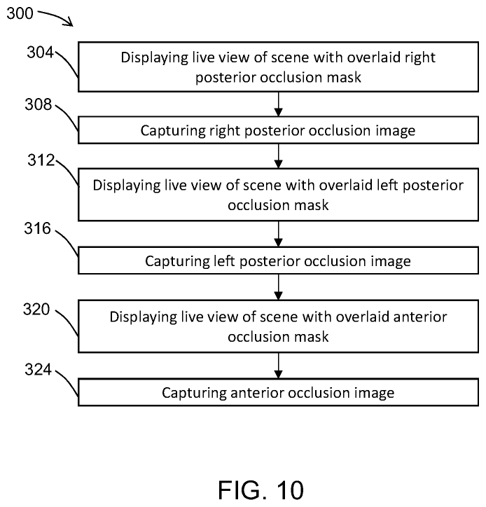

Referring now to FIG. 10, therein illustrated is a flowchart showing the operational steps of a method 300 for capturing a set of occlusion images for a given subject.

At step 304, the live view of the scene captured by the camera is displayed while also displaying the overlaid right posterior occlusion mask.

At step 308, in response to receiving a user-provided capture command, the instant scene is captured and becomes the right posterior occlusion image.

At step 312, the live view of the scene captured by the camera is displayed while also displaying the overlaid left posterior occlusion mask.

At step 316, in response to receiving a user-provided capture command, the instant scene is captured and becomes the left posterior occlusion image.

At step 320, the live view of the scene captured by the camera is displayed while also displaying the overlaid anterior occlusion mask.

At step 324, in response to receiving a user-provided capture command, the instant scene is captured and becomes the anterior occlusion image.

The occlusion classification system 100 and method described herein according to various example embodiments can take on different computer-based implementations.

In one network-based implementations, the occlusion image(s) of the subject are taken using a user device associated to the subject, such as a mobile device (smartphone, tablet, laptop, etc.) or a desktop-based device. The user device can run an application (ex: mobile application, web-based application, or desktop application) that guides the user to capture the occlusion image(s) as described elsewhere herein (ex: the image capture module 172). Upon capturing the occlusion images, these images can be transmitted over a suitable communication network (ex: the Internet) to a server. Various other modules, including the image processing/feature extraction module 112, the occlusion classification neural network(s) and the interpolation module 196 can be implemented at the server, which determines the occlusion class indicator(s) as the numerical output value(s) and/or the recommend treatment.

These outputted values can be further transmitted by the server to one or more devices associated to other parties that are involved in the orthodontic treatment of the subject. For example, the outputted values can be transmitted to one or more of orthodontic professionals that could offer the treatment (orthodontist, dentist, technician, etc) and insurance company covering the costs of the orthodontic treatment.

According to another example implementation, the occlusion classification system 100 can be wholly implemented on the user device. More particularly, each of the image capture module 172, the image processing/feature extraction module 112, the occlusion classification neural network(s) 124 and the interpolation module 196 are implemented on the user device. It will be appreciated that the user device, which may be a mobile device, has limited available computing resources. Therefore, the occlusion classification neural network 124 has to be sufficiently lightweight so that it can be implemented using these limited computing resources. It was observed that the occlusion classification neural network 124 having the RBF architecture present one such implementation that is sufficiently lightweight to allow the occlusion classification system 100 to be wholly implemented on the user device. The person operating the user device can then choose to transmit the output values and recommended treatment to other parties related to the orthodontic treatment.

### Experimental Data

In one experimental implementation, a posterior occlusion classification neural network was trained using a posterior occlusion training dataset and an anterior occlusion classification neural network was trained using an anterior occlusion training dataset. The posterior occlusion training database contained 1693 images of right and left poses, and 289 validation database images. The images are sorted within three classes, namely Class I, Class II and Class III. The reduced input vector dimension from the raw image through principal component analysis (PCA) yielded 50 features of interest. The clustering algorithm on the other hand yielded 13 centres with its corresponding standard deviations as initial values for the training algorithm, therefore leading to 13 RBF as a unique layer.

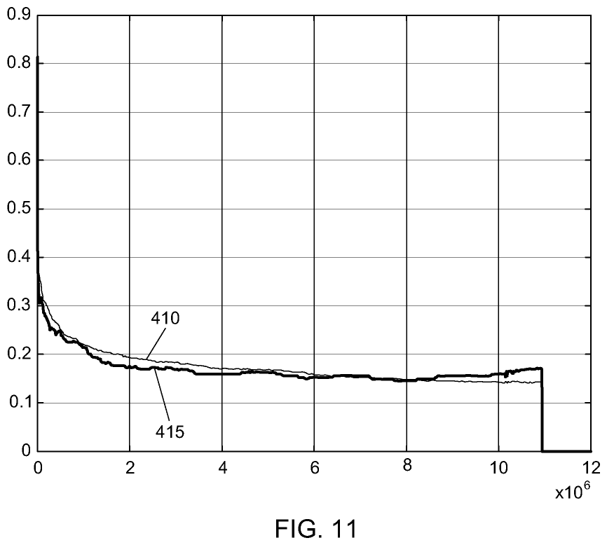

The machine learning method applied to the posterior occlusion neural network is based on the gradient-descent approach, and is simultaneously applied to the centres, their standard deviations, and the weights W1. As shown in FIG. 11, 11 million iterations where performed for training (training curve 410) and the optimal point on the validation data was obtained at about 8 million iterations and corresponds initially to an accuracy rate of 85.5% (validation curve 415). FIG. 11 illustrates a chart showing the posterior occlusion machine learning error reduction, with the training dataset 420 and the validation dataset 425.

The anterior occlusion training database contained 330 images of right and left poses, and 120 validation database images. The images are sorted within three classes, namely ordinary anterior occlusion, open bite and deep bite. The reduced input vector dimension from the raw image through PCA yielded 50 features of interest. The clustering algorithm yielded 6 centres with its corresponding standard deviations as initial values for the training algorithm, therefore leading to 6 RBF as a unique layer.

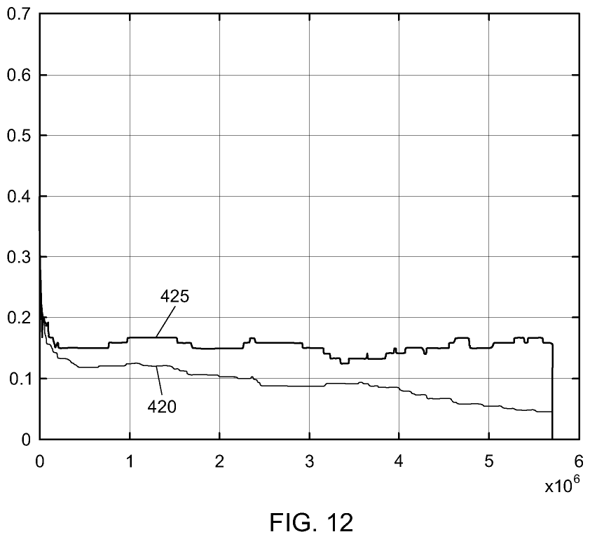

The machine learning method applied to the anterior occlusion neural network is also based on the gradient-descent approach, and is simultaneously applied to the centres, their standard deviations, and the weights. Almost 6 million iterations were performed for training (training curve, blue) and the optimal point on the validation data was obtained at about 3.3 million iterations and corresponds initially to an accuracy rate of 87.5% (validation curve, red). FIG. 12 illustrates a chart showing the anterior occlusion machine learning error reduction, with the training dataset in blue and validation dataset in red.

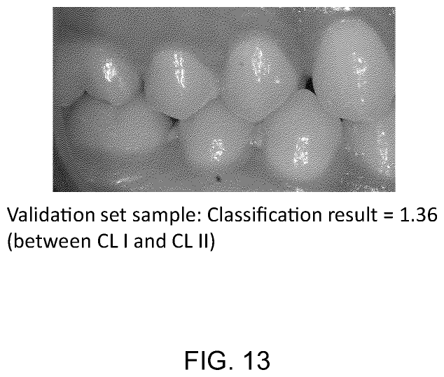

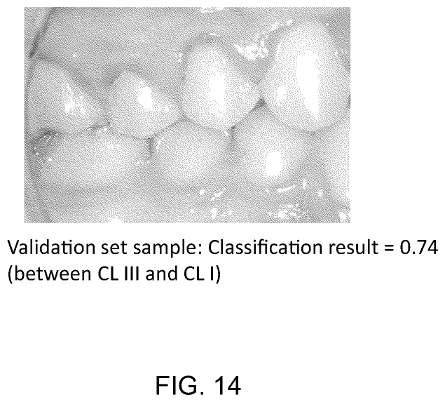

The experimental implementation validated the following observations. The use of the first, second and third numerical values for each class of a training dataset, with each numerical value for each class being representative of where that given class falls within a spectrum of occlusion conditions allowed for training each classification neural network to be operable to predict a numerical output value that is within a continuous range of values. That numerical output value in the continuous range is indicative of where the occlusion image falls within the spectrum of possible occlusion conditions. Moreover, a combination of numerical output values for each of an anterior occlusion image, a left posterior occlusion image and a right posterior occlusion image allows for inter-class interpolation of these values when determining the recommend treatment. FIG. 13 shows a first posterior occlusion image classified by the experimentally implemented posterior occlusion classification neural network. The occlusion image was classified as having a numerical output value of 1.36, which indicates that the occlusion is between a Class I and Class II posterior occlusion. FIG. 14 shows a second posterior occlusion image classified by the experimentally implemented posterior occlusion classification neural network. The occlusion image was classified as having a numerical output value of 0.74, which indicates that the occlusion is between a Class III and Class I posterior occlusion.

It was also observed that the classification neural network having the RBF architecture provided good performance even when trained using small training datasets. This increases access of the solution for enterprises that have less resources (i.e. limited access to large training datasets). The RBF architecture allows for accessible training by machine learning of the classification neural network without requiring extensive computing resources. This can result in lower costs during development.

The RBF network can also, to some extent, be able to detect if an image input is invalid (either a bad framing or an image not related to orthodontic photos in our case) In FIG. 2, the outputs Ri from 140 (centres) are normalized numbers between 0 and 1. Suppose a trained neural network is being used on posterior occlusion images. When a posterior occlusion image is inputted to the network, there is a good probability that this image will be close to one of the centres of the network. Therefore, the correspondent R would be a relatively high number (higher than a certain threshold). Therefore, if, for example the maximum of all the Ri is taken for a specific image and this maximum is relatively high, then it can be deduced that the image is a posterior occlusion image because it is at least near on of the centres Ci. However, if all Ri for a specific image are relatively low (lower than the abovementioned threshold), this means that this image is far from all centres, and is likely not to be related to a posterior occlusion image, (or it could be a bad framing). So, this is a simple means for detection.

The occlusion classification system 100 and method described herein according to various example embodiments can be applied in the following practical applications:
Advertising of dental clinics in the form of a short video before diagnosis is obtained by the subject;
Selling subject information (name, age, address, geolocation, date of birth, photos obtained) and data to a dental clinic able to treat the subject depending on the difficulty of the case. The dental clinic pays a fee in return of the referral, after obtaining the subject's consent. The dental clinic determines the types and ages of subjects it wishes to accept in its office depending on its experience.
Before accepting the treatment plan, an insurance company can use the application to determine whether or not the case can be accepted for payment. The application and software format can be integrated into the insurance company's application and the inclusion criteria would be modified to meet the specific requirements of the insurance company.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A method for determining at least one occlusion class indicator corresponding to at least one occlusion image, the method comprising:
acquiring the at least one occlusion image of an occlusion of a human subject by an image capture device;
applying at least one computer-implemented occlusion classification neural network to the at least one occlusion image to determine the at least one occlusion class indicator of the occlusion of the human subject, the at least one occlusion classification neural network being trained for classification using at least one occlusion training dataset, each given at least one occlusion training dataset including a plurality of occlusion training examples being pre-classified into one of at least:
a first occlusion class, being attributed a first numerical value for the given occlusion type training dataset;
a second occlusion class, being attributed a second numerical value for the given occlusion type training dataset;
a third occlusion class, being attributed a third numerical value for the given occlusion type training dataset, wherein the first numerical value is between the second numerical value for the given occlusion type training dataset and the third numerical value for the given occlusion type training dataset;
each occlusion training example comprising:
a respective training occlusion image, being input data; and
its respective numerical value, being output data;
wherein the at least one occlusion class indicator of the occlusion of the human subject determined by the at least one computer-implemented occlusion classification neural network includes at least one numerical output value within a continuous range of values having the second numerical value as a first bound and the third numerical value as a second bound.

2. The method of claim 1, wherein the image capture device is comprised in a mobile device running a mobile application.

3. The method of claim 1, wherein the at least one occlusion classification neural network comprises an anterior occlusion classification neural network;
wherein the at least one occlusion training dataset comprises an anterior occlusion training dataset for training the anterior occlusion classification neural network, the plurality of occlusion training examples of the anterior occlusion training dataset being pre-classified into at least:
an ordinary anterior occlusion class, representing the first occlusion class and being attributed the first numerical value for the anterior occlusion training dataset;
an open bite occlusion class, representing the second occlusion class and being attributed the second numerical value for the anterior occlusion training dataset;
a deep bite occlusion class, representing the third occlusion class and being attributed the third numerical value for the anterior occlusion training dataset; and
wherein the at least one occlusion class indicator of the occlusion of the human subject includes an anterior occlusion numerical output value determined by the anterior occlusion classification neural network, the anterior occlusion numerical output value being in the continuous range of values having the second numerical value for the anterior occlusion training dataset as a first bound and the third numerical value for the anterior occlusion training dataset as a second bound.

4. The method of claim 1, wherein the at least occlusion classification neural network comprises a posterior occlusion classification neural network;
wherein the at least one occlusion training dataset comprises a posterior occlusion training dataset for training the posterior occlusion classification neural network, the plurality of occlusion training examples of the posterior occlusion training dataset being pre-classified into at least:
a class I posterior occlusion class, representing the first occlusion class and being attributed the first numerical value for the posterior occlusion training dataset;
a class II posterior occlusion class, representing the second occlusion class and being attributed the second numerical value for the posterior occlusion training dataset;
a class III posterior occlusion class, representing the third occlusion class and being attributed the third numerical value for the posterior occlusion training dataset;
wherein the at least one occlusion class indicator of the occlusion of the human subject includes a posterior occlusion numerical output value determined by the posterior occlusion classification neural network, the posterior occlusion numerical output value being in the continuous range of values having the second numerical value for the posterior occlusion training dataset as a first bound and the third numerical value for the posterior occlusion training dataset as a second bound.

5. The method of claim 1, wherein the at least one occlusion classification neural network comprises an anterior occlusion classification neural network and a posterior occlusion classification neural network;
wherein the at least one occlusion training dataset comprises an anterior occlusion training dataset for training the anterior occlusion classification neural network and a posterior occlusion training dataset for training the posterior occlusion classification neural network;
wherein the plurality of occlusion training examples of the anterior occlusion training dataset is pre-classified into at least:
an ordinary anterior occlusion class, representing the first occlusion class and being attributed the first numerical value for the anterior occlusion training dataset;
an open bite occlusion class, representing the second occlusion class and being attributed the second numerical value for the anterior occlusion training dataset;
a deep bite occlusion class, representing the third occlusion class and being attributed the third numerical value for the anterior occlusion training dataset; and
wherein the at least one occlusion class indicator of the occlusion of the human subject includes an anterior occlusion numerical output value determined by the anterior occlusion classification neural network, the anterior occlusion numerical output value being in a first continuous range of values having the second numerical value for the anterior occlusion training dataset as a first bound and the third numerical value for the anterior occlusion training dataset as a second bound;
wherein the plurality of occlusion training examples of the posterior occlusion training dataset is pre-classified into at least:
a class I posterior occlusion class, representing the first occlusion class and being attributed the first numerical value for the posterior occlusion training dataset;
a class II posterior occlusion class, representing the second occlusion class and being attributed the second numerical value for the posterior occlusion training dataset;
a class III posterior occlusion class, representing the third occlusion class and being attributed the third numerical value for the posterior occlusion training dataset;
wherein the at least one occlusion class indicator of the occlusion of the human subject includes a posterior occlusion numerical output value determined by the posterior occlusion classification neural network, the posterior occlusion numerical output value being in the continuous range of values having the second numerical value for the posterior occlusion training dataset as a first bound and the third numerical value for the posterior occlusion training dataset as a second bound.

6. The method of claim 5, wherein the at least one occlusion image of the human subject comprises a left posterior occlusion image, a right posterior occlusion image, and an anterior occlusion image;
wherein the posterior occlusion classification neural network is applied to the left posterior occlusion image to determine a left posterior occlusion numerical output value;
wherein the posterior occlusion classification neural network is applied to the right posterior occlusion image to determine a right posterior occlusion numerical output value; and
wherein the anterior occlusion classification neural network is applied to the anterior occlusion image to determine the anterior occlusion numerical output value.

7. The method of claim 6, wherein the at least one occlusion class indicator further comprises an interpolation of at least two output values selected from the group consisting of the left posterior occlusion numerical output value, the right posterior occlusion numerical output value and the anterior numerical output value.

8. The method of claim 6, further comprising cropping and normalizing the at least one occlusion image of the occlusion of the human subject prior to applying the at least one computer-implemented occlusion classification neural network thereto.

9. The method of claim 8, wherein cropping the at least one occlusion image is performed semi-automatically using at least one overlaid mask.

10. The method of claim 9, wherein acquiring the at least one occlusion image comprises:
displaying a live view of a first scene and a left posterior occlusion mask overlaid on the live view of the first scene;
in response to a first capture command, capturing a first image corresponding to the first scene, the first image being the left posterior occlusion image of the at least one occlusion image of the occlusion of the human subject;
displaying a live view of a second scene and a right posterior occlusion mask overlaid on the live view of the second scene;
in response to a second capture command, capturing a second image corresponding to the second scene, the second image being the right posterior occlusion image of the at least one occlusion image of the occlusion of the human subject;
displaying a live view of a third scene and an anterior occlusion mask overlaid on the live view of the third scene; and
in response to a third capture command, capturing a third image corresponding to the third scene, the third image being the anterior occlusion image of the at least one occlusion image of the occlusion of the human subject.

11. The method of claim 1, wherein the at least one computer-implemented occlusion classification neural network comprises at least one radial basis function neural network.

12. The method of claim 11, wherein applying the at least one radial basis function neural network comprises extracting a feature vector from each of the at least one occlusion image.

13. The method of claim 12, wherein extracting the feature vector comprises applying a principal component analysis to each of the at least one occlusion image.

14. The method of claim 12, wherein the at least one radial basis function neural network is configured to receive the feature vector.

15. (canceled)

16. The method of claim 11, wherein the at least one radial basis function neural network has between approximately 10 centres and approximately 20 centres.

17. The method of claim 16, further comprising determining that a given one of the at least one occlusion image is an inappropriate occlusion image based on the given occlusion image being greater than a threshold distance from each of the centres.

18. The method of claim 1, further comprising diagnosing an orthodontic malocclusion.

19. The method of claim 7, further comprising determining a treatment for an orthodontic malocclusion.

20. A system for determining at least one occlusion class indicator, the system comprising:
at least one data storage device storing executable instructions;
at least one processor coupled to the at least one storage device, the at least one processor being configured to execute the instructions and thereby to perform a method of comprising:
acquiring the at least one occlusion image of an occlusion of a human subject by an image capture device;
applying at least one computer-implemented occlusion classification neural network to the at least one occlusion image to determine the at least one occlusion class indicator of the occlusion of the human subject, the at least one occlusion classification neural network being trained for classification using at least one occlusion training dataset, each given at least one occlusion training dataset including a plurality of occlusion training examples being pre-classified into one of at least:
a first occlusion class, being attributed a first numerical value for the given occlusion type training dataset;
a second occlusion class, being attributed a second numerical value for the given occlusion type training dataset;
a third occlusion class, being attributed a third numerical value for the given occlusion type training dataset, wherein the first numerical value is between the second numerical value for the given occlusion type training dataset and the third numerical value for the given occlusion type training dataset;
each occlusion training example comprising:
a respective training occlusion image, being input data; and
its respective numerical value, being output data;
wherein the at least one occlusion class indicator of the occlusion of the human subject determined by the at least one computer-implemented occlusion classification neural network includes at least one numerical output value within a continuous range of values having the second numerical value as a first bound and the third numerical value as a second bound.

21. A computer program product comprising a computer readable memory storing computer executable instructions thereon that when executed by a computer perform a method comprising:
acquiring the at least one occlusion image of an occlusion of a human subject by an image capture device;
applying at least one computer-implemented occlusion classification neural network to the at least one occlusion image to determine the at least one occlusion class indicator of the occlusion of the human subject, the at least one occlusion classification neural network being trained for classification using at least one occlusion training dataset, each given at least one occlusion training dataset including a plurality of occlusion training examples being pre-classified into one of at least:
a first occlusion class, being attributed a first numerical value for the given occlusion type training dataset;
a second occlusion class, being attributed a second numerical value for the given occlusion type training dataset;
a third occlusion class, being attributed a third numerical value for the given occlusion type training dataset, wherein the first numerical value is between the second numerical value for the given occlusion type training dataset and the third numerical value for the given occlusion type training dataset;
each occlusion training example comprising:
a respective training occlusion image, being input data; and
its respective numerical value, being output data;
wherein the at least one occlusion class indicator of the occlusion of the human subject determined by the at least one computer-implemented occlusion classification neural network includes at least one numerical output value within a continuous range of values having the second numerical value as a first bound and the third numerical value as a second bound.
